(19) United States
(12) Patent Application Publication
(10) Pub. No.: US 2025/0117930 A1
(43) Pub. Date: Apr. 10, 2025

(54) MEDICAL IMAGE PROCESSING DEVICE AND MEDICAL IMAGE PROCESSING METHOD

(71) Applicants: CANON KABUSHIKI KAISHA, Tokyo (JP); CANON MEDICAL SYSTEMS CORPORATION, Tochigi (JP)

(72) Inventors: Naoki MATSUKI, Tokyo (JP); Yuko TAKADA, Tochigi (JP)

(21) Appl. No.: 18/903,015

(22) Filed: Oct. 1, 2024

(30) Foreign Application Priority Data
Oct. 5, 2023 (JP) 2023-173727
Jul. 12, 2024 (JP) 2024-112578

(51) Int. Cl. G06T 7/00; G16H 50/20

(52) U.S. Cl. CPC G06T 7/0012; G16H 50/20; G06T 2207/30096

(57) ABSTRACT

A reference region useful for inference of diagnosis information is acquired from a medical image and used for inference of diagnosis information. Provided is a medical image processing device including: a medical image acquisition unit that acquires a medical image including at least a region of a tumor mass; an auxiliary information acquisition unit that acquires auxiliary information including at least any one of A) image finding information, which is based on the medical image and is information about a predetermined image finding representing a nature of the tumor mass, and B) first diagnosis information, which is based on the medical image and is information about diagnosis of the tumor mass; and a diagnosis information inference unit that infers second diagnosis information, which is information about diagnosis of the tumor mass, in response to input of an image generated based on the medical image and on the auxiliary information.

### BACKGROUND OF THE INVENTION

### Field of the Invention

The disclosure of the present specification relates to a medical image processing device and a medical image processing method.

### Description of the Related Art

Nowadays, various types of medical information are utilized for diagnosis, and there is an increasing demand for systems used by users such as physicians to use results obtained through analysis by a computer or the like on medical information such as medical images as an aid in diagnosis.

A document titled “The uncertainty of boundary can improve the classification accuracy of BI-RADS 4A ultrasound image” (Huayu Wang et al., 2022) (PubMed) discloses a method of using a tumor mass region together with the original image when acquiring diagnosis information on the tumor mass in an ultrasound image of a mammary gland. Further, Japanese Patent Application Laid-Open No. 2019-191772 discloses a method of adjusting a region used for inference in accordance with the type of a finding to be acquired when acquiring an image finding of a node in a computer tomography (CT) image of a lung.

When inferring diagnosis information, it is possible to obtain a reliable inference result by preferentially using auxiliary information such as information on a region to be referenced together with an image. In the methods disclosed in Japanese Patent Application Laid-Open No. 2019-191772 and “The uncertainty of boundary can improve the classification accuracy of BI-RADS 4A ultrasound image” (Huayu Wang et al., 2022) (PubMed), however, a region useful for inference of diagnosis information, such as a region in which an image finding appears in an image may be excluded from a reference region. Further, in the methods disclosed in Japanese Patent Application Laid-Open No. 2019-191772 and “The uncertainty of boundary can improve the classification accuracy of BI-RADS 4A ultrasound image” (Huayu Wang et al., 2022) (PubMed), a region in which no image finding appears and that is unnecessary for inference of diagnosis information may be included in a reference region.

### SUMMARY OF THE INVENTION

Provided is a medical image processing device including:
a medical image acquisition unit that acquires a medical image including at least a region of a tumor mass;
an auxiliary information acquisition unit that acquires auxiliary information including at least any one of
   A) image finding information, which is based on the medical image and is information about a predetermined image finding representing a nature of the tumor mass, and
   B) first diagnosis information, which is based on the medical image and is information about diagnosis of the tumor mass; and
a diagnosis information inference unit that infers second diagnosis information, which is information about diagnosis of the tumor mass, in response to input of an image generated based on the medical image and on the auxiliary information.

Further features of the present invention will become apparent from the following description of exemplary embodiments with reference to the attached drawings.

### BRIEF DESCRIPTION OF THE DRAWINGS

FIG. 1 illustrates an example of a hardware configuration of a medical image processing device according to embodiments of the present invention.

FIG. 2 illustrates an example of the function configuration of the medical image processing device according to the embodiments of the present invention.

FIG. 3 is a flowchart illustrating an example of the process in the medical image processing device according to the embodiments of the present invention.

FIG. 4A illustrates an example of an ultrasound image.

FIG. 4B illustrates an example of a tumor mass region according to the embodiments of the present invention.

FIG. 4C illustrates an example of a region near a tumor mass as an example of the tumor mass region according to the embodiments of the present invention.

FIG. 5A illustrates an example of an indistinct region of a tumor mass margin as an example of auxiliary information according to a first embodiment of the present invention.

FIG. 5B illustrates an example of a spicula region of a tumor mass margin as an example of the auxiliary information according to the first embodiment of the present invention.

FIG. 5C illustrates an example of a rough region of a tumor mass margin as an example of the auxiliary information according to the first embodiment of the present invention.

FIG. 5D illustrates an example of a bright portion inside the tumor mass as an example of the auxiliary information according to the first embodiment of the present invention.

FIG. 6 illustrates an example of a reference region according to the embodiments of the present invention.

FIG. 7A illustrates an example of two-channel one-image input as an example of a configuration of a diagnosis information inference unit according to the embodiments of the present invention.

FIG. 7B illustrates an example of two-image input as an example of a configuration of the diagnosis information inference unit according to the embodiments of the present invention.

FIG. 7C illustrates an example of one-channel one-image input as an example of a configuration of the diagnosis information inference unit according to the embodiments of the present invention.

FIG. 8A illustrates an example displaying diagnosis information as an example of a display method in the medical image processing device according to the embodiments of the present invention.

FIG. 8B illustrates an example displaying diagnosis information and a reference region as an example of the display method in the medical image processing device according to the embodiments of the present invention.

FIG. 8C illustrates an example displaying diagnosis information and a finding region as an example of the display method in the medical image processing device according to the embodiments of the present invention.

FIG. 9 is a flowchart illustrating an example of the process in a medical image processing device according to a second embodiment of the present invention.

FIG. 10A illustrates an example of a correspondence table of specific processes for auxiliary information and tumor mass regions according to the second embodiment of the present invention.

FIG. 10B illustrates an example of a correspondence table of specific processes for auxiliary information and tumor mass regions according to the second embodiment of the present invention.

FIG. 11 illustrates an example of a reference region according to the embodiments of the present invention.

FIG. 12 illustrates an example of a reference region according to the embodiments of the present invention.

FIG. 13A illustrates an example of three-channel one-image input as an example of the configuration of the diagnosis information inference unit according to the embodiments of the present invention.

FIG. 13B illustrates an example of three-image input as an example of the configuration of the diagnosis information inference unit according to the embodiments of the present invention.

FIG. 14 illustrates an example of the configuration of the diagnosis information inference unit according to the embodiments of the present invention.

FIG. 15 illustrates an example of the function configuration of a medical image processing device according to a fourth embodiment of the present invention.

FIG. 16 is a flowchart illustrating an example of the process in the medical image processing device according to the fourth embodiment of the present invention.

FIG. 17A illustrates an example of a correspondence table of specific modification processes for auxiliary information and medical images according to the fourth embodiment of the present invention.

FIG. 17B illustrates an example of a correspondence table of specific modification processes for auxiliary information and medical images according to the fourth embodiment of the present invention.

FIG. 18 illustrates an example of a gradation mask according to the fourth embodiment of the present invention.

FIG. 19A illustrates an example of the configuration of the diagnosis information inference unit according to the fourth embodiment of the present invention.

FIG. 19B illustrates an example of the configuration of the diagnosis information inference unit according to the fourth embodiment of the present invention.

FIG. 20 illustrates an example of the function configuration of a medical image processing device according to Modified example 1 for the fourth embodiment of the present invention.

FIG. 21 illustrates an example of a tumor mass internal region, a tumor mass margin region, and a tumor mass peripheral region according to the fourth embodiment of the present invention.

FIG. 22 illustrates an example of the function configuration of a medical image processing device according to Modified example 3 for the fourth embodiment of the present invention.

### DESCRIPTION OF THE EMBODIMENTS

The embodiments according to the present invention provide the following medical image processing device.

A medical image processing device including:
a medical image acquisition unit that acquires a medical image including at least a region of a tumor mass;
an auxiliary information acquisition unit that acquires auxiliary information including at least any one of
   A) image finding information, which is based on the medical image and is information about a predetermined image finding representing a nature of the tumor mass, and
   B) first diagnosis information, which is based on the medical image and is information about diagnosis of the tumor mass; and
a diagnosis information inference unit that infers second diagnosis information, which is information about diagnosis of the tumor mass, in response to input of an image generated based on the medical image and on the auxiliary information.

The image finding information of A) may be information about an image finding region in a medical image, that is, about an image (first embodiment) or may be other information than the image based on an image finding (second embodiment). An example of the information about an image may be information about the presence or absence, the type, or the degree of an image finding. Note that the image finding refers to, for example, the distinctness of the margin of a tumor mass, the roughness of the margin of a tumor mass, a linear opacity (for example, a spicula) of the margin of a tumor mass, or the like.

The first diagnosis information of B) (third embodiment) may be a result of image diagnosis, a result of pathological diagnosis, or the like obtained from a medical image, and may more specifically be a category such as the BI-RADS or the JABTS, the grade of malignancy of a tumor mass, or the pathology of a tumor mass.

The diagnosis information inference unit can have a reference region acquisition unit that acquires a reference region image in the medical image as the generated image based on the medical image and on the auxiliary information. In this case, the diagnosis information inference unit can infer the second diagnosis information based on the reference region image.

The reference region acquisition unit can acquire a region enlarged from or a region reduced from the region of the tumor mass as a reference region based on the auxiliary information. In this case, it is possible to determine a ratio of enlargement or reduction of the region of the tumor mass based on the auxiliary information. The reference region acquisition unit can further adjust the sharpness of the boundary of the acquired reference region based on the auxiliary information. Furthermore, the reference region acquisition unit can include a first reference region acquisition unit that acquires a first reference region in the medical image based on the medical image and on the auxiliary information and a second reference region acquisition unit that acquires a second reference region, which differs from the first reference region, based on the medical image and the auxiliary information. In this case, the diagnosis information inference unit can infer the second diagnosis information on the tumor mass based on the medical image, on the first reference region, and on the second reference region. The diagnosis information inference unit can have a first region-related diagnosis information inference unit that infers first region-related diagnosis information, which is diagnosis information about the first reference region, based on the medical image and on the first reference region and a second region-related diagnosis information inference unit that infers second region-related diagnosis information, which is diagnosis information about the second reference region, based on the medical image and on the second reference region and can infer the second diagnosis information based on the first region-related diagnosis information and on the second region-related diagnosis information.

The reference region acquisition unit may acquire a region enlarged from the region of the tumor mass as a reference region so as to include an image finding region.

The medical image processing device may include an input image acquisition unit that acquires, as an image generated based on the medical image and on the auxiliary information, an image obtained by modifying a pixel value of the medical image based on the auxiliary information. In this case, the diagnosis information inference unit can infer the second diagnosis information based on the input image.

The input image acquisition unit may acquire a medical image processed so as to emphasize at least any one of an inside, a margin, or a periphery of the tumor mass in accordance with a result of the auxiliary information.

The input image acquisition unit may acquire a medical image processed so as to reduce at least any one of an inside, a margin, or a periphery of the tumor mass in accordance with a result of the auxiliary information.

The auxiliary information may include at least the image finding information of A), the image finding information may include information about an image finding region that is a region in the medical image in which the image finding is present, and the input image acquisition unit may acquire a medical image processed so as to emphasize the inside or the outside of the image finding region.

The auxiliary information may include at least the image finding information of A), the image finding information may include information about an image finding region that is a region in the medical image in which the image finding is present, and the input image acquisition unit may acquire a medical image processed so as to reduce the inside or the outside of the image finding region.

The first diagnosis information can include information about at least any one of: a result of pathological diagnosis on the tumor mass; and a grade of malignancy of the tumor mass based on a result of image diagnosis on the tumor mass, and the second diagnosis information can include information about the grade of malignancy of the tumor mass.

The image finding information can include information about an image finding region that is a region in the medical image in which the image finding is present. Further, the image finding information can include information about the presence or absence, a type, or a degree of the image finding. The image finding information can include at least any one of information about distinctness of a margin of the tumor mass, information about roughness of a margin of the tumor mass, and information about the presence or absence of a linear opacity of a margin of the tumor mass.

The embodiments according to the present invention will be described below in detail with reference to the attached drawings. Note that the embodiments disclosed as examples below are not intended to limit the present invention recited in the claims, and not all the combinations of features described in the present embodiments are necessarily required for the solutions of the present invention.

### First Embodiment

The first embodiment is, in particular, a medical image processing device in which the auxiliary information includes the image finding information of A), the image finding information includes information about an image finding region that is a region in the medical image in which the image finding is present, the diagnosis information inference unit has a reference region acquisition unit that acquires a reference region image in the medical image based on the medical image and on the auxiliary information and infers the second diagnosis information based on the reference region image. In the following example, the image finding information can include at least any one of information about the distinctness of the margin of a tumor mass, information about the roughness of the margin of a tumor mass, and information about the presence or absence of a linear opacity of the margin of a tumor mass. In the example below, in particular, an ultrasound image of a mammary gland is used as a medical image (which may be simply referred to as an image), and a case where the image finding information is an image finding region including a finding about the distinctness of the margin of a tumor mass will be described as an example.

### Hardware Configuration

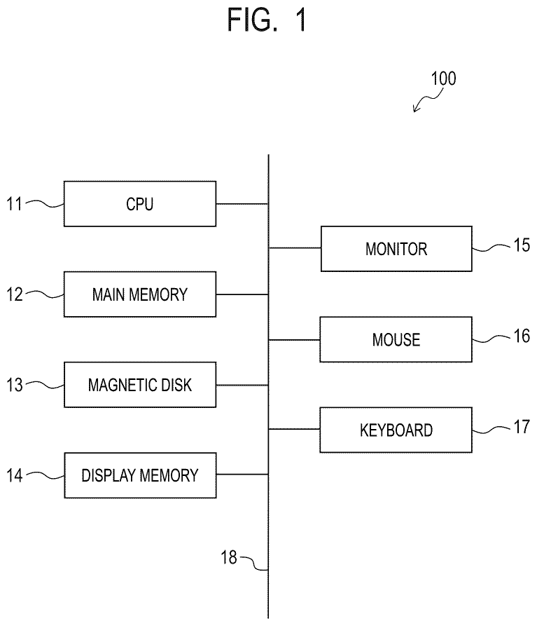

FIG. 1 is a diagram illustrating an example of a hardware configuration of a medical image processing device in the present embodiment. A CPU 11 mainly controls the operation of each component. A main memory 12 stores a control program executed by the CPU 11 and provides a working area used when the CPU 11 is executing a program. A magnetic disk 13 stores operating system (OS), device drivers for peripheral devices, and programs used for implementing various application software including a program used for performing the process described later or the like. The CPU 11 executes the program stored in the main memory 12 or the magnetic disk 13, and thereby the function (software) of the medical image processing device in the present embodiment is implemented.

A display memory 14 temporarily stores displaying data to be displayed on a monitor 15, for example. The monitor 15 is, for example, a CRT monitor, a liquid crystal monitor, or the like and displays images, texts, or the like based on data from the display memory 14. A mouse 16 and a keyboard 17 are used by the user to perform pointing input and input of characters or the like, respectively.

These components described above are communicably connected to each other via a common bus 18.

Note that the configuration of the medical image processing device 100 is not limited to the above. For example, the medical image processing device 100 may have a plurality of processors. Further, the medical image processing device 100 may have a GPU or a field-programmable gate array (FPGA) in which a part of the process is programmed.

### Function Configuration

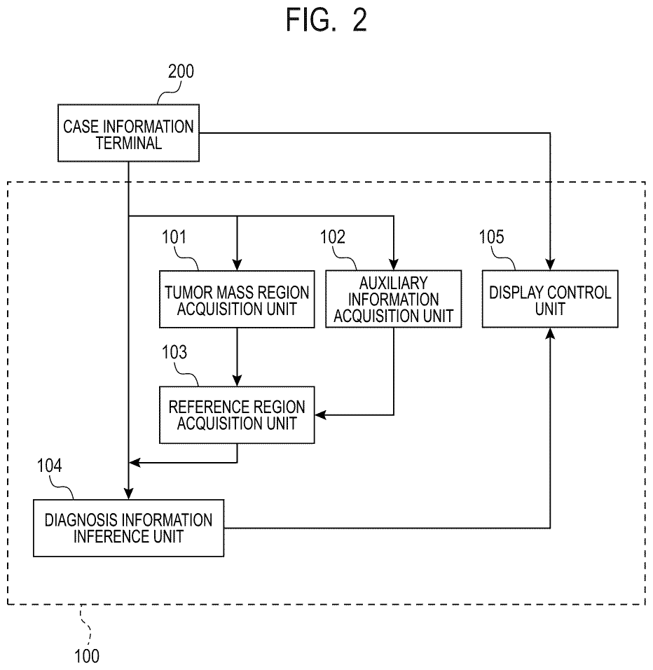

FIG. 2 is a diagram illustrating an example of the function configuration of the medical image processing device in the present embodiment.

The medical image processing device 100 is communicably connected to a case information terminal 200. The medical image processing device 100 has a tumor mass region acquisition unit 101, an auxiliary information acquisition unit 102, a reference region acquisition unit 103, a diagnosis information inference unit 104, and a display control unit 105. These function configurations of the medical image processing device 100 are connected to each other via an internal bus or the like.

The case information terminal 200 acquires information about a case to be diagnosed from a server (not illustrated). For example, the information about a case is medical information such as clinical information described in a medical image or an electronic medical record. For example, the case information terminal 200 may be connected to an ultrasound image diagnosis device to acquire ultrasound images based on a physician's operation on a probe. Further, for example, the case information terminal 200 may be connected to an external storage device (not illustrated) such as an HDD, a solid state drive (SSD), a CD drive, or a DVD drive to acquire medical images from these external storage devices.

Further, the case information terminal 200 provides, via the display control unit 105, a GUI that allows the user to select one of the acquired medical images. The selected medical image is displayed on the monitor 15 in an enlarged manner. The case information terminal 200 transmits a medical image selected via the GUI by the user to the medical image processing device 100 via a network or the like. Note that ultrasound images acquired in real time based on the physician's operation on the probe may be sequentially transmitted to the medical image processing device 100 instead of the medical image to be transmitted to the medical image processing device 100 being selected by the user.

Note that the case information terminal 200 may provide, via the display control unit 105, a GUI that allows the user to designate a rectangular region surrounding a tumor mass in a medical image and transmit a cutout image, which corresponds to a region selected by the user via the GUI and is cut out from the medical image, to the medical image processing device 100 as a medical image. Note that a reasoner that infers a rectangular region surrounding a tumor mass may be built in advance through deep learning or the like based on an image, and the rectangular region may be automatically acquired by using the reasoner without allowing the user to designate a rectangular region surrounding the tumor mass.

Based on a medical image transmitted from the case information terminal 200 to the medical image processing device 100 (hereafter, referred to as a target image), the tumor mass region acquisition unit 101 acquires a region of a tumor mass (hereafter, referred to as a tumor mass region) included in the target image.

The auxiliary information acquisition unit 102 acquires auxiliary information based on the target image transmitted from the case information terminal 200 to the medical image processing device 100.

The reference region acquisition unit 103 acquires a reference region based on the tumor mass region acquired by the tumor mass region acquisition unit 101 and on the auxiliary information acquired by the auxiliary information acquisition unit 102.

The diagnosis information inference unit 104 infers diagnosis information on a tumor mass included in a target image based on the target image transmitted from the case information terminal 200 to the medical image processing device 100 and on the reference region acquired by the reference region acquisition unit 103.

The display control unit 105 displays diagnosis information acquired by the diagnosis information inference unit 104 on the monitor 15 together with a medical image and a GUI.

### Process Flow

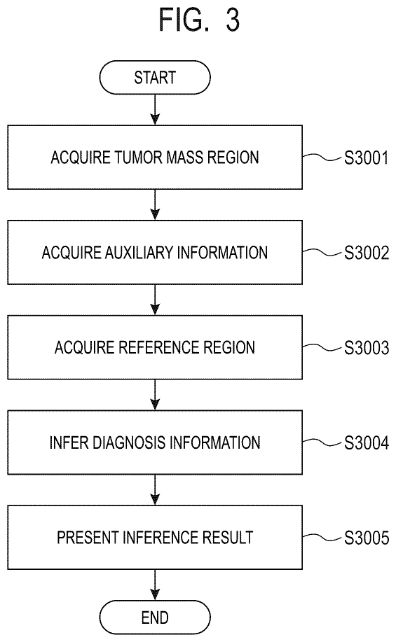

FIG. 3 is a flowchart illustrating an example of the process performed by the medical image processing device 100. In the first embodiment, the CPU 11 executes programs implementing the functions of respective units stored in the main memory 12, and thereby the process illustrated in FIG. 3 is realized.

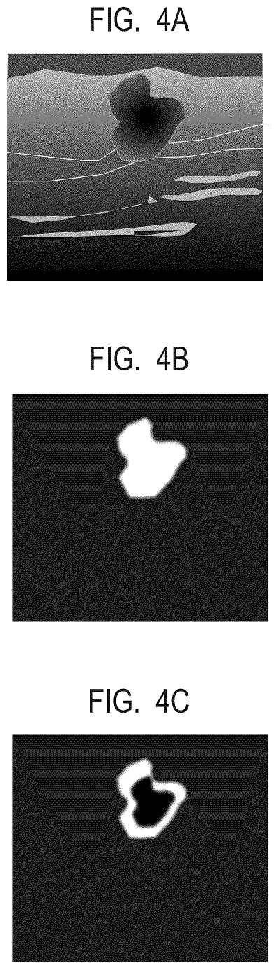

In step S3001, based on a target image transmitted from the case information terminal 200 to the medical image processing device 100 (an example of an ultrasound image is illustrated in FIG. 4A as the target image), the tumor mass region acquisition unit 101 acquires a tumor mass region (illustrated in FIG. 4B as an example) included in the target image. To acquire a tumor mass region, a reasoner built in advance through deep learning or the like to infer tumor mass regions based on target images is used. Note that the configuration of the tumor mass region acquisition unit 101 is not limited to the above, and the tumor mass region acquisition unit 101 may be configured to acquire a tumor mass region for the target image from an external server (not illustrated) that provides the same function. Further, the tumor mass region acquisition unit 101 may be configured to provide, via the display control unit 105, the GUI that allows the user to designate any region in the target image to allow the user to designate a tumor mass region.

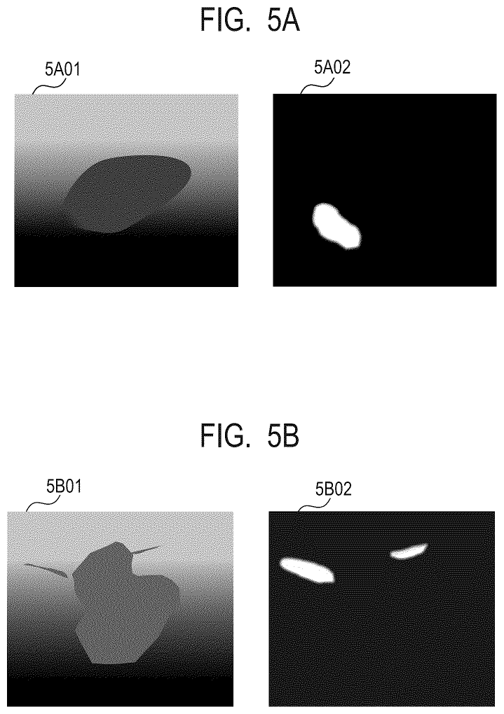

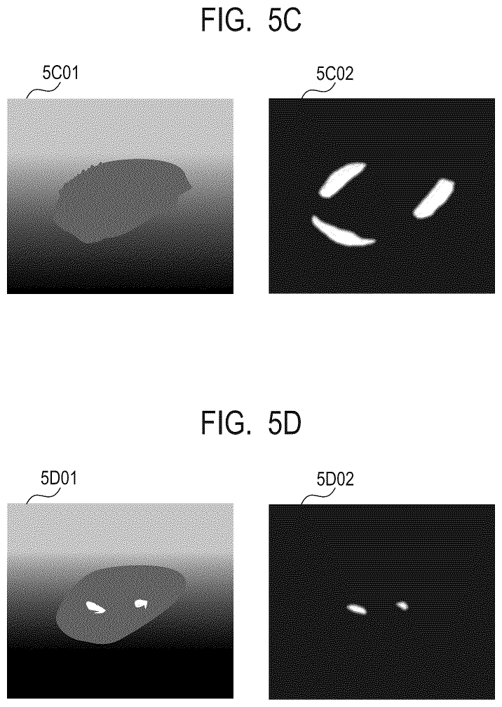

In step S3002, based on the target image transmitted from the case information terminal 200 to the medical image processing device 100, the auxiliary information acquisition unit 102 acquires a region of a predetermined image finding (hereafter, referred to as a finding region) included in the target image as the auxiliary information. The predetermined image finding in the present embodiment is the presence of an indistinct tumor mass margin, and a region of an indistinct portion of a tumor mass margin is acquired as the finding region. FIG. 5A illustrates an example of a region of an indistinct portion of a tumor mass margin. In FIG. 5A, a finding region image 5A02 represents a region of an indistinct portion of the margin of a tumor mass present in an ultrasound image 5A01. The finding region image 5A02 is a binary image expressed by two types of: a foreground region represented in white; and a background region represented in black in the drawing, and the foreground region represents a region of an indistinct portion of the margin of a tumor mass. Note that the finding region image 5A02 is not necessarily required to be a binary image and may have continuous values, and an intermediate region may thus be present in which the foreground region and the background region are mixed. To acquire a finding region, a reasoner built in advance through deep learning or the like to infer the finding region based on images is used. Note that the configuration of the auxiliary information acquisition unit 102 is not limited to the above, and the auxiliary information acquisition unit 102 may be configured to acquire a finding region by using an image processing scheme which is not based on machine learning. For example, the auxiliary information acquisition unit 102 may be configured to focus on a margin portion of a tumor mass region in a target image and determine that a region whose edge intensity is lower than a threshold is the finding region, for example. Further, the auxiliary information acquisition unit 102 may be configured to acquire a finding region for a target image from an external server (not illustrated) that provides the same function. Further, the auxiliary information acquisition unit 102 may be configured to provide, via the display control unit 105, the GUI that allows the user to designate any region in a target image to allow the user to designate a finding region.

Further, the input to the reasoner is not limited to the target image. For example, the method may be configured to input the target image and the tumor mass region acquired in step S3001 (the tumor mass region acquisition unit 101) together to the reasoner. In this case, the reasoner is also built so as to infer the finding region based on the image and on the tumor mass region.

Further, the method may be configured to exclude, out of the finding region, a region of a portion where the distance from the boundary of the tumor mass region acquired in step S3001 (the tumor mass region acquisition unit 101) exceeds a predefined threshold. In this case, the threshold is designated by a ratio or the like when the number of pixels or the width or the height of a rectangular region surrounding a tumor mass region is defined as 1.

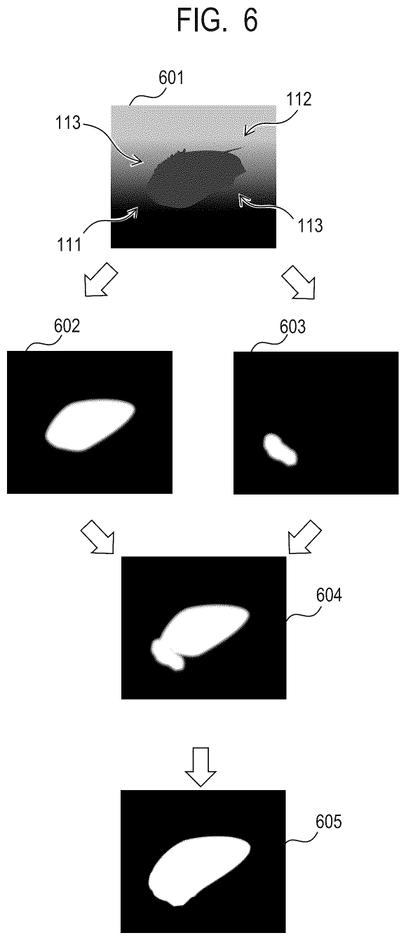

In step S3003, the reference region acquisition unit 103 acquires a reference region based on the tumor mass region acquired in step S3001 (the tumor mass region acquisition unit 101) and the auxiliary information acquired in step S3002 (the auxiliary information acquisition unit 102). In the present embodiment, a reference region is the sum set of a tumor mass region and a finding region acquired as auxiliary information. Note that a reference region may be acquired by a known smoothing process so that the boundaries of the tumor mass region and the finding region are smoothly connected to each other. FIG. 6 illustrates an example of the reference region. In FIG. 6, a tumor mass region image 602 represents a region of a tumor mass present in an ultrasound image 601. A finding region image 603 represents a region of an indistinct portion of the margin of the tumor mass present in the ultrasound image 601. The ultrasound image 601 represents an indistinct region 111, a spicula region 112, and a rough region 113 as examples of findings. A reference region image 604 represents a reference region acquired as the sum set of the tumor mass region image 602 and the finding region image 603. A reference region image 605 represents a reference region acquired so that the boundaries of the tumor mass region image 602 and the finding region image 603 are smoothly connected to each other.

The reference region image 604 and the reference region image 605 are binary images expressed by two types of: the foreground region represented in white; and the background region represented in black in the drawing, and the foreground region represents a region intended to be specifically referenced when diagnosis information is inferred. By inputting a target image and a reference region together to the diagnosis information inference unit 104, it is possible to transfer the information on a region intended to be specifically referenced in the target image to the diagnosis information inference unit 104. Accordingly, compared to a case where only the target image is input, the foreground region of the reference region is expected to be preferentially used for inference of diagnosis information by the diagnosis information inference unit 104. Note that the reference region is not necessarily required to be a binary image and may have continuous values, and an intermediate region may thus be present in which the foreground region and the background region are mixed.

In step S3004, the diagnosis information inference unit 104 infers diagnosis information on a tumor mass included in the target image based on the target image transmitted from the case information terminal 200 to the medical image processing device 100 and the reference region acquired in step S3003 (the reference region acquisition unit 103). In the present embodiment, the BI-RADS category of a tumor mass (for example, described in ACR BI-RADS ATLAS 5th Edition (American College of Radiology)) is inferred as diagnosis information. To infer diagnosis information, a reasoner built in advance through deep learning or the like to infer diagnosis information based on both an image and a region is used. Note that the configuration of the diagnosis information inference unit 104 is not limited to the above, and the diagnosis information inference unit 104 may be configured to transmit a target image and a reference region to an external server (not illustrated) that provides the same function as the diagnosis information inference unit 104, cause the server to infer diagnosis information, and acquire the diagnosis information.

Further, the target to be inferred as the diagnosis information is not limited to the BI-RADS category of a tumor mass. For example, any index representing the grade of malignancy of a tumor mass, such as the JABTS category (for example, Guidelines for Breast Ultrasound Diagnosis, Revised 3rd Edition (Japanese Association of Breast and Thyroid Sonology)) may be inferred as the diagnosis information. Further, the grade of malignancy based on a result of pathological diagnosis may be inferred as the diagnosis information. Further, the pathology of a tumor mass (cyst, fibroadenoma, adenocarcinoma, or the like) may be inferred as the diagnosis information.

Further, the diagnosis information may be a category value or may be a likelihood of each category. Further, the diagnosis information may be continuous values representing the grade of malignancy (for example, values when complete benignancy is defined as 0 and complete malignancy is defined as 1).

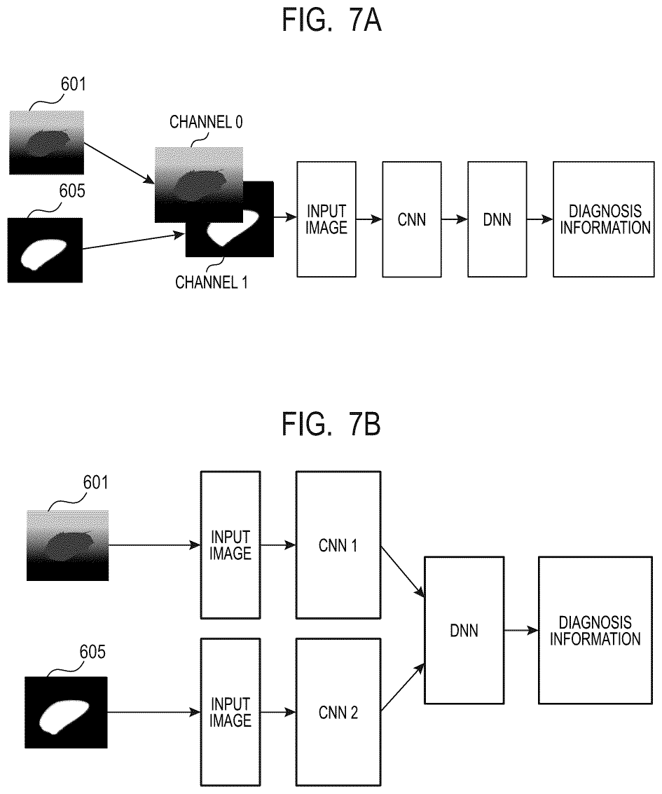

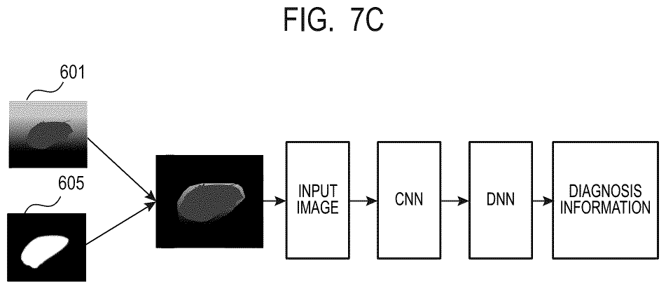

In the present embodiment, for the reasoner, as illustrated in FIG. 7A as an example, a reasoner having a configuration in which a convolutional neural network (CNN) and a deep neural network (DNN) are combined is built and used in which the CNN performs a convolutional process on images in response to receiving two-channel image data as the input and the DNN infers diagnosis information in response to receiving the output of the CNN as the input. A target image (ultrasound image) and a reference region are integrated into one image data with different channels and then input to the reasoner.

Further, as illustrated in FIG. 7B as an example, the reasoner may be configured such that a CNN1 that receives a target image (ultrasound image) as the input and a CNN2 that receives a reference region as the input are separately prepared, and the CNN1 and CNN2 are combined with a DNN that infers diagnosis information in response to receiving the output of the CNN1 and the output of the CNN2 as the input.

Further, as illustrated in FIG. 7C as an example, the reasoner may be configured such that a CNN that performs convolutional process on images in response to receiving one-channel image data as the input is combined with a DNN that infers diagnosis information in response to receiving the output of the CNN as the input. For example, the image input to the reasoner illustrated in FIG. 7C as an example is input to the reasoner as one-channel image data after a mask process is performed on a target image (ultrasound image) using a reference region.

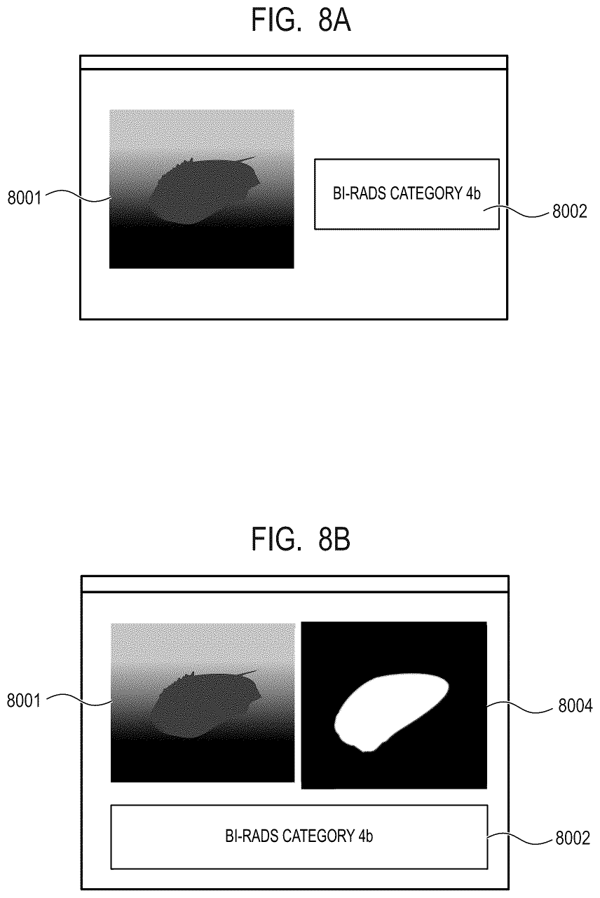

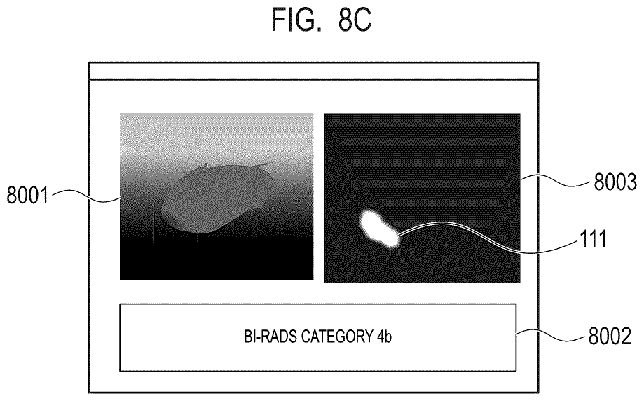

In step S3005, the display control unit 105 displays the diagnosis information 8002 acquired in step S3004 (the diagnosis information inference unit 104) on the monitor 15 together with medical image 8001 including a tumor mass image and a GUI in this example (illustrated in FIG. 8A as an example). Further, the display control unit 105 may additionally display the reference region 8004 acquired in step S3003 (the reference region acquisition unit 103) (illustrated in FIG. 8B as an example). Further, the display control unit 105 may additionally display the auxiliary information acquired in step S3002 (the auxiliary information acquisition unit 102) (illustrated in FIG. 8C as an example). In the example of FIG. 8C, an indistinct region 111 is displayed as a finding region 8003. Further, the display control unit 105 may additionally display the tumor mass region acquired in step S3001 (the tumor mass region acquisition unit 101).

Note that the process of step S3005 (process of displaying diagnosis information) is not necessarily required. For example, the method may be configured to store or output diagnosis information in or to a storage device or the like (not illustrated) without displaying the diagnosis information.

The process described above enables automatic acquisition of a reference region including a finding region in a medical image and thus enables the use of the reference region for inference of diagnosis information. Accordingly, a finding region in a medical image can be caused to be preferentially referenced when diagnosis information is inferred, and a reliable inference result can be obtained.

### Modified Example 1 for First Embodiment

The image finding information may be, for example, information about distinctness of the margin of a tumor mass, information about the roughness of the margin of a tumor mass, and information about the presence or absence of a linear opacity of the margin of a tumor mass. In step S3002, the finding region acquired as the auxiliary information by the auxiliary information acquisition unit 102 is not limited to the region of an indistinct portion of the tumor mass margin illustrated in FIG. 5A. For example, a spicula region that is a linear opacity of a tumor mass margin may be acquired as the auxiliary information. FIG. 5B illustrates a finding region image 5B02 of a spicula of the margin of a tumor mass included in an ultrasound image 5B01 as an example. Further, for example, the region of a rough portion of the tumor mass boundary may be acquired as the auxiliary information. FIG. 5C illustrates a finding region image 5C02 of a rough portion of the margin of a tumor mass included in an ultrasound image 5C01 as an example. Further, for example, a bright region inside a tumor mass may be acquired as the auxiliary information. Further, a combination of these regions may be acquired as the auxiliary information. FIG. 5D illustrates a finding region image 5D02 of a bright portion inside a tumor mass included in an ultrasound image 5D01 as an example.

Accordingly, a finding region effective for inference can be selectively included in a reference region in accordance with a target to be inferred by the diagnosis information inference unit 104.

For example, when determination of whether or not the tumor mass is obviously malignant (for example, BI-RADS category “5”) is acquired as the diagnosis information in step S3004, the region 111 of the indistinct portion of the tumor mass margin, the region 112 of the spicula of the tumor mass margin, and the region 113 of the rough portion of the tumor mass margin are acquired as the auxiliary information in step S3002. These are regions of important findings in determining whether or not the tumor mass is malignant. By acquiring a reference region (illustrated in FIG. 11 as an example) including these finding regions in step S3003 and inputting the reference region together with the target image to the diagnosis information inference unit 104 in step S3004, it is possible to cause the diagnosis information inference unit 104 to reference the region of the finding effective in determining whether or not the tumor mass is malignant.

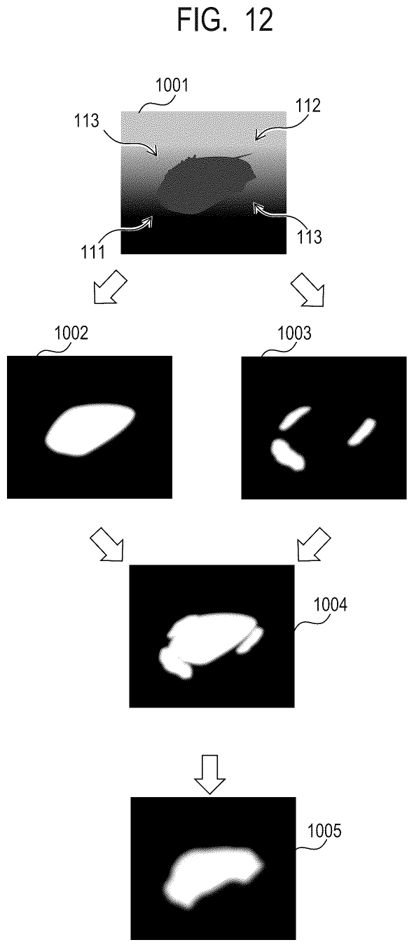

Further, for example, when determination of whether or not the tumor mass is obviously benign (for example, BI-RADS category “2”) is acquired as the diagnosis information in step S3004, the region 111 of the indistinct portion of the tumor mass margin and the region 113 of the rough portion of the tumor mass margin are acquired as the auxiliary information in step S3002. In determining whether or not the tumor mass is benign, it is important that there is no region of these findings (that the tumor mass margin is clear and smooth (Circumscribed)). By acquiring a reference region (illustrated in FIG. 12 as an example) including these finding regions in step S3003 and inputting the reference region together with the target image to the diagnosis information inference unit 104 in step S3004, it is possible to cause the diagnosis information inference unit 104 to reference the region of the finding effective in determining whether or not the tumor mass is benign. Note that, in FIG. 11 and FIG. 12, “1001” represents an ultrasound image, “1002” represents a tumor mass region image, “1003” represents a finding region image, “1004” represents a reference region image, and “1005” represents a reference region image (after a smoothing process).

### Modified Example 2 for First Embodiment

In step S3002, the finding region acquired as the auxiliary information by the auxiliary information acquisition unit 102 is not limited to those included in a tumor mass or those in contact with a tumor mass. For example, a region in which a finding related to the condition where a mammary gland expands to the entire image (duct dilatation, architectural distortion, or the like) appears may be acquired as the auxiliary information.

For example, in step S3002, a region in which the breast duct expands in an image is acquired as the auxiliary information. The information on the expansion of the breast duct serves as information for making determination as to whether the image feature found in a tumor mass region (the inside or marginal part of a tumor mass) is a localized feature of the tumor mass region or the condition of the breast duct is merely viewed as if it were a feature of the tumor mass region, and this information is effective in determining diagnosis information. By acquiring a reference region so that a region in which a breast duct expands is included in the reference region in step S3003 and inputting the reference region together with the target image to the diagnosis information inference unit 104 in step S3004, it is possible to cause the diagnosis information inference unit 104 to reference the region of the finding effective in determining diagnosis information.

### Modified Example 3 for First Embodiment

In step S3001, the tumor mass region acquired by the tumor mass region acquisition unit 101 may be such a region that covers only the margin of a tumor mass (illustrated in FIG. 4C as an example). In this case, the width of the region is adaptively determined in accordance with the size of the tumor mass, such as being determined in accordance with the width of the tumor mass in a medical image.

Accordingly, in step S3004, the diagnosis information inference unit 104 can cause the reasoner to preferentially reference the information on the boundary and the margin of a tumor mass that is particularly important when inferring diagnosis information on the tumor mass.

### Modified Example 4 for First Embodiment

In step S3004, the reference region input to the reasoner by the diagnosis information inference unit 104 may be modified in advance.

For example, a blurring process with predefined parameters may be applied to a reference region. This makes it possible that the reasoner is less likely to be affected by a fine feature of the external shape of the reference region.

### Modified Example 5 for First Embodiment

In the present modified example, the reference region acquisition unit includes a first reference region acquisition unit that acquires a first reference region in a medical image and a second reference region acquisition unit that acquires a second reference region that differs from the first reference region, and the diagnosis information inference unit infers the second diagnosis information on a tumor mass based on the medical image, on the first reference region, and on the second reference region.

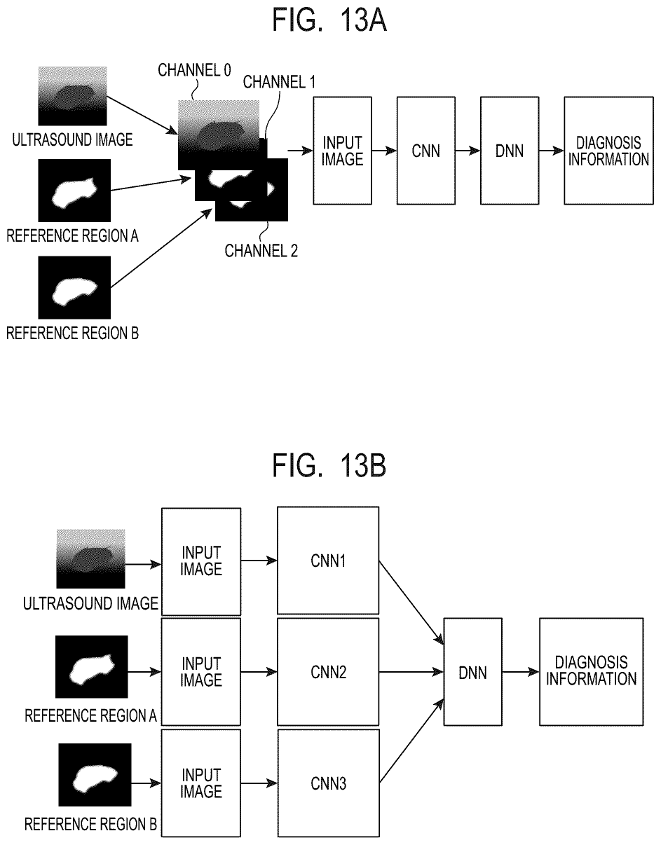

That is, the diagnosis information inference unit 104 can be configured to infer diagnosis information by using a plurality of different reference regions in step S3004. For example, as illustrated in FIG. 13A and FIG. 13B, the diagnosis information inference unit 104 may be configured to infer the diagnosis information by using a reference region A and a reference region B. Herein, FIG. 13A represents an example of a reasoner configured such that a CNN that performs a convolutional process on images in response to receiving three-channel image data as the input is combined with a DNN that infers diagnosis information in response to receiving the output of the CNN as the input, and a target image (ultrasound image), the reference region A, and the reference region B are integrated into one image data with different channels and then input to the reasoner. FIG. 13B represents an example of a reasoner configured such that a CNN1 that receives a target image (ultrasound image) as the input, a CNN2 that receives the reference region A as the input, and a CNN3 that receives the reference region B as the input are separately prepared and combined with a DNN that infers diagnosis information in response to receiving the output of the CNN1 and the output of the CNN2 and CNN3 as the input.

Herein, for example, a region of an indistinct portion of a tumor mass margin, a region of a spicula of a tumor mass margin, and a region of a rough portion of a tumor mass margin are acquired as the auxiliary information in step S3002, and the reference region A is acquired as the reference region 1004 which includes these pieces of auxiliary information (illustrated in FIG. 11 as an example) in step S3003. Further, for example, a region of an indistinct portion of a tumor mass margin and a region of a rough portion of a tumor mass margin are acquired in step S3002, and the reference region B is acquired as the reference region 1004 which includes these pieces of auxiliary information (illustrated in FIG. 12 as an example) in step S3003.

When there are multiple types of regions effective in determining diagnosis information, the above process can cause the diagnosis information inference unit 104 to reference respective types of such regions.

### Modified Example 6 for First Embodiment

The diagnosis information inference unit is formed of a plurality of reasoners and can acquire the final diagnosis information. In this example, the auxiliary information may include only one piece of auxiliary information, that is, only the first auxiliary information or may include multiple pieces of auxiliary information, that is, the first auxiliary information and the second auxiliary information. Further, the reference region may include only one reference region, that is, only the first reference region or may include a plurality of reference regions, that is, the first reference region and the second reference region. This will be more specifically described below.

In step S3004, the diagnosis information inference unit 104 can be configured to combine inference units with each other that infer multiple pieces of different diagnosis information to acquire the final diagnosis information.

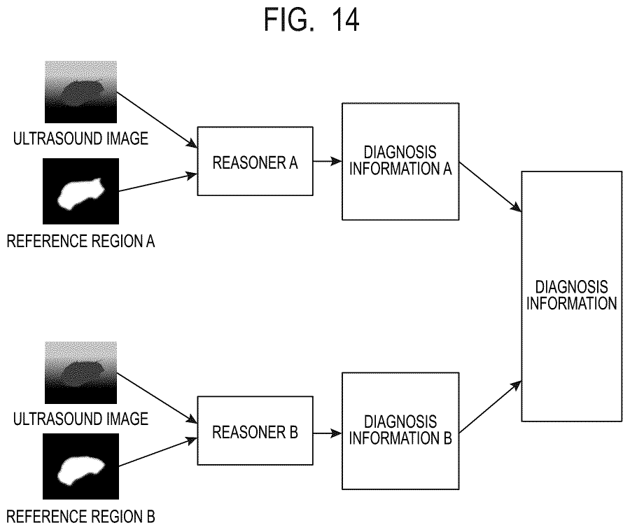

For example, as illustrated in FIG. 14, a reasoner A that acquires, as diagnosis information A, information about whether or not the tumor mass is categorized into the BI-RADS category “5” and a reasoner B that acquires, as diagnosis information B, information about whether or not the tumor mass is categorized into the BI-RADS category “2” may be combined, and information about which of the BI-RADS category “5”, the BI-RADS category “2”, or “other” the tumor mass is categorized into can be acquired as the final diagnosis information.

In this case, the pieces of auxiliary information input to the reasoner A and reasoner B may differ from each other. For example, for the reasoner A, a region of an indistinct portion of the tumor mass margin, a region of a spicula of the tumor mass margin, and a region of a rough portion of the tumor mass margin may be acquired as the auxiliary information (first auxiliary information) in step S3002, and the reference region 1004 which includes the first auxiliary information (the first reference region, illustrated in FIG. 11 as an example) may be acquired in step S3003. Further, for example, for the reasoner B, a region of an indistinct portion of the tumor mass margin and a region of a rough portion of the tumor mass margin may be acquired as the auxiliary information (second auxiliary information) in step S3002, and the reference region 1004 which includes the auxiliary information (the second reference region, illustrated in FIG. 12 as an example) may be acquired in step S3003. Then, in step S3004, by inputting the first reference region to a discriminator A together with the target image, it is possible to cause the discriminator A to reference the region of the finding effective in determining whether or not the tumor mass is malignant. Further, similarly, by inputting the second reference region to a discriminator B together with the target image, it is possible to cause the discriminator B to reference the region of the finding effective in determining whether or not the tumor mass is benign.

When there are multiple types of regions effective in determining diagnosis information, the above process can cause the diagnosis information inference unit 104 to reference respective types of such regions.

### Modified Example 7 for First Embodiment

In step S3003, a region obtained by enlarging a tumor mass region so as to include an image finding region can be acquired as a reference region. Alternatively, a tumor mass region can be enlarged or reduced based on auxiliary information, and this enlarged or reduced region can be used as a reference region. In this case, the sharpness of the boundary of the acquired reference region may be adjusted based on auxiliary information. Further, the ratio of enlargement and reduction may be determined based on auxiliary information.

### Second Embodiment

The second embodiment is a medical image processing device characterized in that the auxiliary information includes at least A) the image finding information, and the image finding information includes information about the presence or absence, the type, or the degree of the image finding.

The medical image processing device according to the second embodiment adjusts and acquires a region to be preferentially referenced when inferring diagnosis information in accordance with the content of the finding that can be acquired from an image. Note that the medical image processing device according to the second embodiment is formed of the functions illustrated in FIG. 2 in the same manner as in the first embodiment.

### Process Flow

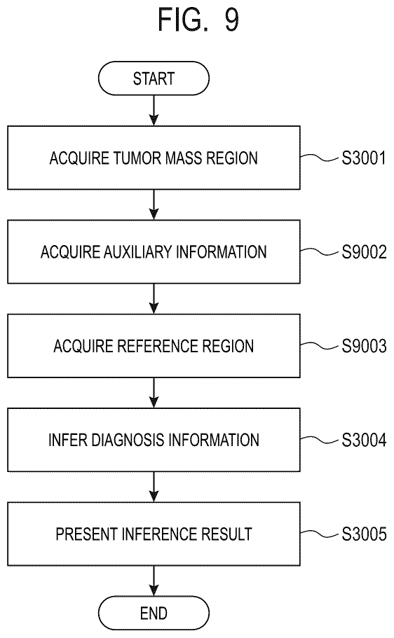

FIG. 9 is a flowchart illustrating an example of the process performed by the medical image processing device 100 according to the second embodiment. Step S3001, step S3004, and step S3005 are the same as those of the first embodiment. In the following, only the features different from those of the first embodiment will be described.

In step S9002, based on a target image transmitted from the case information terminal 200 to the medical image processing device 100, the auxiliary information acquisition unit 102 according to the second embodiment acquires, as auxiliary information, a category value of a predetermined image finding included in the target image. In the present embodiment, the degree of indistinctness of a tumor mass margin is classified into four categories of category 0 (non), category 1 (small), category 2 (medium), and category 3 (large), and a category to which the target image belongs is acquired as the auxiliary information. Note that the configuration of the auxiliary information acquisition unit 102 is not limited to the above, and the auxiliary information acquisition unit 102 may be configured to acquire a finding region related to a target image from an external server (not illustrated) that provides the same function. Further, the auxiliary information acquisition unit 102 may be configured to provide, via the display control unit 105, a GUI used for selecting or inputting a category value of an image finding of a target image to allow the user to designate a category value.

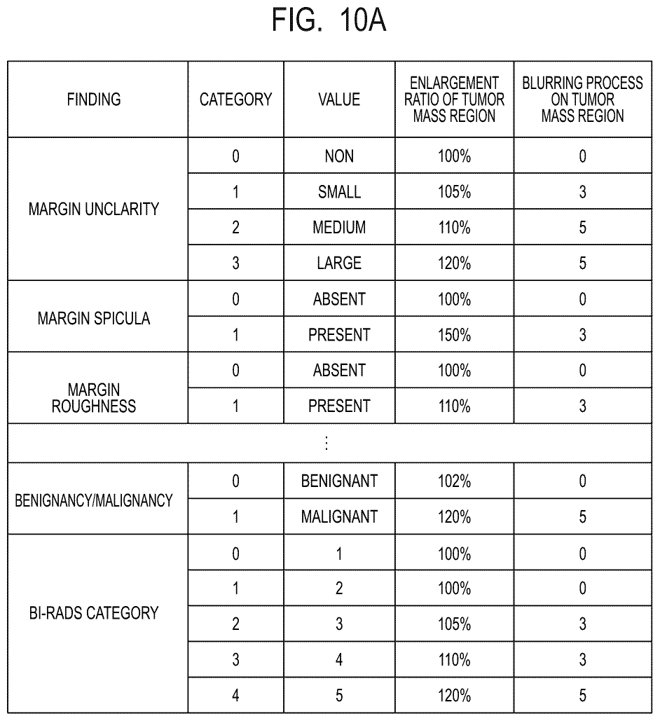

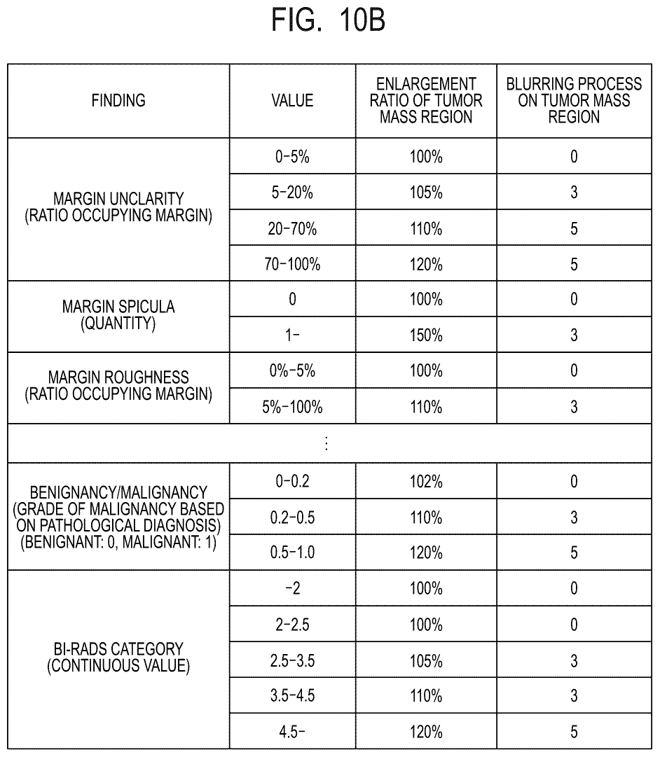

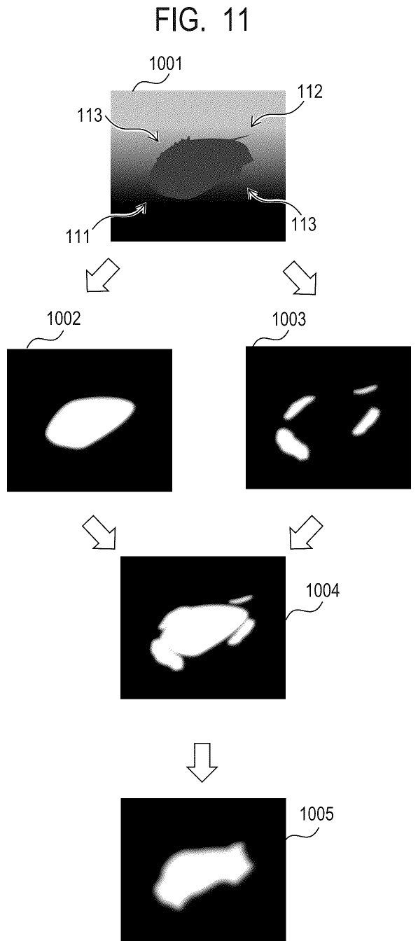

In step S9003, the reference region acquisition unit 103 according to the second embodiment references a correspondence table illustrated in FIG. 10A as an example based on the category value of indistinctness of the tumor mass margin, which is the auxiliary information acquired in step S9002 (the auxiliary information acquisition unit 102) and acquires parameters for a process performed on the tumor mass region. Furthermore, the reference region acquisition unit 103 applies a process to the tumor mass region with the acquired parameters and acquires the processed tumor mass region as a reference region. In the present embodiment, the process on a tumor mass region is to enlarge a region and apply blurring with a known filtering process, and the enlargement ratio and the blur kernel size are acquired as the parameters. For example, when the category of indistinctness of a tumor mass margin is category 3 (large), the tumor mass region is enlarged at an enlargement ratio of 120%, and a blurring filter with a kernel size of 5 is applied. Note that another process may be applied to a tumor mass region. For example, sharpening with a known filtering process may be used, and the sharpening kernel size may be able to be acquired from the correspondence table as a parameter.

The process described above enables automatic acquisition of a reference region so that a finding region in a medical image is included in the reference region and thus enables the use of the reference region for inference of diagnosis information. Accordingly, the reasoner for diagnosis information can be caused to reference a finding region in a medical image, and a reliable inference result can be obtained.

### Modified Example 1 for Second Embodiment

In step S9002, the value of an image finding acquired as the auxiliary information by the auxiliary information acquisition unit 102 is not limited to a category value and may be acquired as continuous values. For example, a case where the entire circumference of a tumor mass margin is indistinct may be defined as 100% and a case where no indistinct portion is present may be defined as 0% to acquire the degree of indistinctness of the tumor mass margin. In this case, in step S9003, the reference region acquisition unit 103 references a correspondence table illustrated in FIG. 10B as an example and acquires parameters for a process applied to the tumor mass region.

Accordingly, a process in accordance with a value can be finely defined for a finding that can express the nature of an image in a continuous manner.

### Modified Example 2 for Second Embodiment

In step S9002, the value of an image finding acquired as the auxiliary information by the auxiliary information acquisition unit 102 is not limited to those representing indistinctness of a tumor mass margin. For example, a category value representing whether or not a spicula is present in a tumor mass margin may be acquired as the auxiliary information. Further, for example, the ratio of a rough portion occupying a tumor mass margin may be acquired as the auxiliary information. Further, for example, the presence or absence of a bright region in a tumor mass may be acquired as the auxiliary information.

In this case, in step S9003, the reference region acquisition unit 103 may acquire parameters for a process on a tumor mass region in accordance with a combination of multiple pieces of auxiliary information. For example, when there is no indistinctness, roughness, or spicula in the tumor mass margin and there is a bright region in the tumor mass, the enlargement ratio of the tumor mass region may be set to 90% to reduce the tumor mass region.

Accordingly, the reasoner can be caused to selectively reference a region of a finding effective for inference in accordance with a target to be inferred by the diagnosis information inference unit 104.

### Modified Example 3 for Second Embodiment

In step S9002, the information acquired as the auxiliary information by the auxiliary information acquisition unit 102 may be a region that is important when acquiring a category value of an image finding (important region). For example, the important region is acquired by a method disclosed in “Grad-CAM: Visual Explanations From Deep Networks via Gradient-Based Localization (arXiv)” when the category value of an image finding is acquired by a reasoner with a CNN.

In this case, the important region is handled in the same manner as the finding region image 603 in FIG. 6, and the reference region image 604 may be acquired as the sum set of the tumor mass region image 602 and the important region.

Accordingly, the reasoner can be caused to selectively reference a region of a finding effective for inference in accordance with a target to be inferred by the diagnosis information inference unit 104.

### Third Embodiment

The third embodiment is a medical image processing device characterized in that, in particular, the auxiliary information includes at least B) the first diagnosis information, which is based on the medical image and is information about diagnosis of the tumor mass. More specifically, B) the first diagnosis information, which is based on the medical image and is information about diagnosis of the tumor mass, is information representing benignancy or malignancy of a tumor mass or diagnosis information.

That is, in the present embodiment, in step S9002, the auxiliary information acquired by the auxiliary information acquisition unit 102 is not limited to a value of an image finding, and diagnosis information on a tumor mass may be acquired.

The diagnosis information on a tumor mass is information representing benignancy or malignancy of the tumor mass diagnosed based on a medical image (hereafter, referred to as image diagnosis information), such as the BI-RADS category or the JABTS category described above, for example. Herein, the image diagnosis information is represented by a category value of the BI-RADS category or a likelihood for each category.

Further, the diagnosis information on a tumor mass is, for example, a result obtained by inferring a pathology inspection result from a medical image (hereafter, referred to as pathology inference information). A reasoner used for inference of the pathology inference information is built by deep learning or the like by using a data set in which a result of pathology inspection and a medical image including a tumor mass to be inspected are paired. Herein, the pathology inference information is a category value of benignancy or malignancy or continuous values representing the grade of malignancy. Note that the pathology of a tumor mass such as “cyst”, “fibroadenoma”, “adenocarcinoma”, or the like may be inferred as the category value without being limited to benignancy or malignancy.

For example, the auxiliary information acquisition unit 102 may be configured such that the auxiliary information acquisition unit 102 acquires pathology inference information as the first diagnosis information and, in step S3004, the diagnosis information inference unit 104 infers image diagnosis information as the second diagnosis information. In this case, in step S9003, the reference region acquisition unit 103 according to the second embodiment acquires parameters for a process on the tumor mass region in accordance with the correspondence tables illustrated in FIG. 10A and FIG. 10B. For example, when the category value of benignancy or malignancy is category 1 (malignant), the tumor mass region is enlarged at an enlargement ratio of 120%, and a blurring filter with a kernel size of 5 is applied.

Further, for example, the auxiliary information acquisition unit 102 may be configured such that the auxiliary information acquisition unit 102 acquires image diagnosis information as the first diagnosis information and, in step S3004, the diagnosis information inference unit 104 infers pathology inference information as the second diagnosis information. In this case, in step S9003, the reference region acquisition unit 103 according to the second embodiment acquires parameters for a process on the tumor mass region in accordance with the correspondence tables illustrated in FIG. 10A and FIG. 10B. For example, when the category value of the BI-RADS category is category 4 (5), the tumor mass region is enlarged at an enlargement ratio of 120%, and a blurring filter of a kernel size of 5 is applied.

Accordingly, even when inference of an image finding is difficult, a reference region effective for inference of diagnosis information can be acquired.

Note that the information acquired as the first diagnosis information and the information acquired as the second diagnosis information may be of the same type.

For example, the auxiliary information acquisition unit 102 may be configured such that the auxiliary information acquisition unit 102 acquires pathology inference information as the first diagnosis information and, in step S3004, the diagnosis information inference unit 104 also infers pathology inference information as the second diagnosis information.

Further, for example, the auxiliary information acquisition unit 102 may be configured such that the auxiliary information acquisition unit 102 acquires image diagnosis information as the first diagnosis information and, in step S3004, the diagnosis information inference unit 104 also infers image diagnosis information as the second diagnosis information.

When the second diagnosis information is inferred, since a reference region adjusted based on the first diagnosis information is used, an inferred result reflecting information on a region that is more intended to be referenced than the first diagnosis information can be obtained.

### Fourth Embodiment

As the fourth embodiment, the present invention provides a medical image processing device including an input image acquisition unit that acquires an input image based on a medical image and on auxiliary information. In the present embodiment, the diagnosis information inference unit infers the second diagnosis information, which is information about diagnosis of a tumor mass, based on an input image. In the present embodiment, without requiring acquisition of a reference region, an input image can be acquired based on auxiliary information, and diagnosis information on a tumor mass can be inferred based on the input image.

### Function Configuration

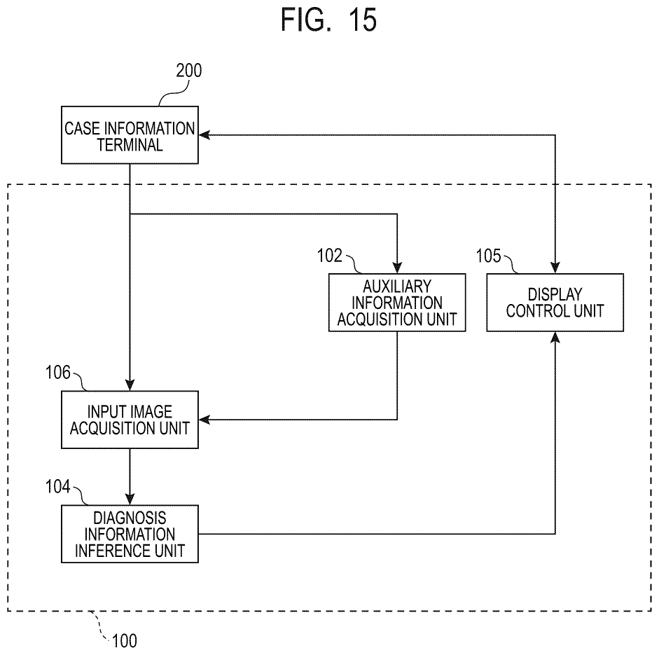

FIG. 15 is a diagram illustrating an example of the function configuration of the medical image processing device in the fourth embodiment. The medical image processing device includes an input image acquisition unit 106 in addition to the function configuration of the first embodiment. Further, the tumor mass region acquisition unit 101 and the reference region acquisition unit 103 are excluded from the function configuration of the first embodiment. Note that features other than the functions described below are the same as those described in the first embodiment.

The input image acquisition unit 106 acquires an input image based on a target image transmitted from the case information terminal 200 to the medical image processing device 100 and on auxiliary information acquired by the auxiliary information acquisition unit 102.

The diagnosis information inference unit 104 in the present embodiment infers diagnosis information on a tumor mass included in the target image based on the input image acquired by the input image acquisition unit 106.

### Process Flow

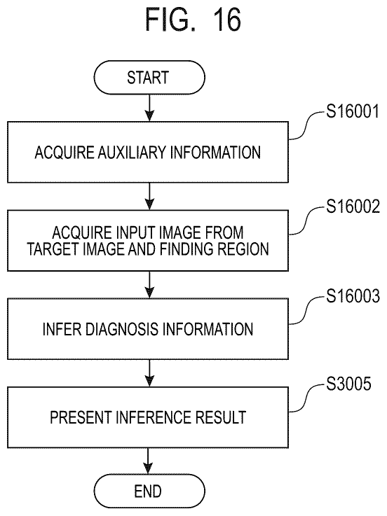

FIG. 16 is a flowchart illustrating an example of the process performed by the medical image processing device 100 in the fourth embodiment. Step S3005 is the same as that of the first embodiment. Only the features different from those of the first embodiment will be described below.

In step S16001, based on a target image transmitted from the case information terminal 200 to the medical image processing device 100, the auxiliary information acquisition unit 102 according to the fourth embodiment acquires, as the auxiliary information, a category value of a predetermined image finding included in the target image. In the present embodiment, the degree of indistinctness of a tumor mass margin is classified into four categories of category 0 (non), category 1 (small), category 2 (medium), and category 3 (large), and a category to which the target image belongs is acquired as the auxiliary information. Furthermore, the presence or absence of a spicula of the tumor mass margin and the presence or absence of a roughness of the tumor mass margin are classified into two categories of category 0 (absent) and category 1 (present), respectively, and a category to which the target image belongs is acquired as the auxiliary information. Furthermore, the state of internal echo pattern is classified into four categories of category 0 (low echo), category 1 (equal echo), category 2 (high echo), and category 3 (uneven), and a category to which the target image belongs is acquired as the auxiliary information. Note that the configuration of the auxiliary information acquisition unit 102 is not limited to the above, and the auxiliary information acquisition unit 102 may be configured to acquire a category value of an image finding of a target image from an external server (not illustrated) that provides the same function. Further, the auxiliary information acquisition unit 102 may be configured to provide, via the display control unit 105, a GUI used for selecting or inputting a category value of an image finding of a target image to allow the user to designate a category value.

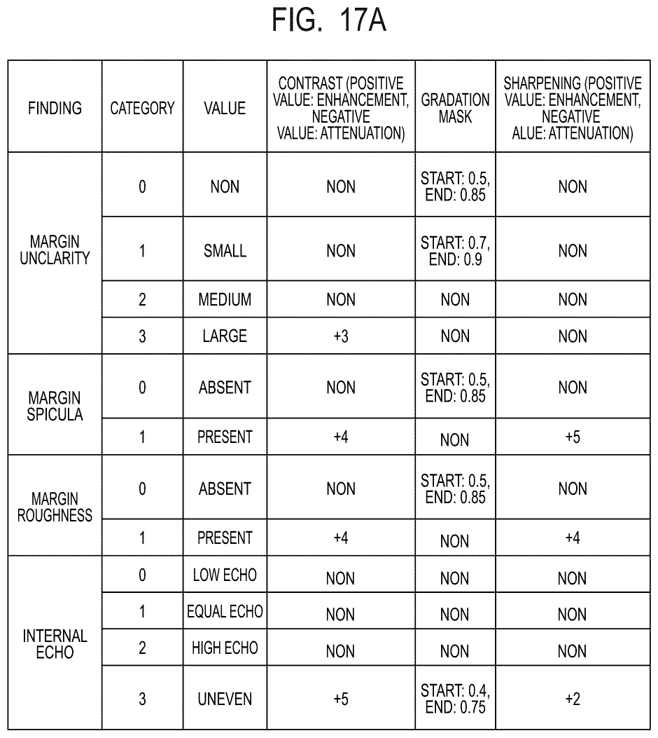

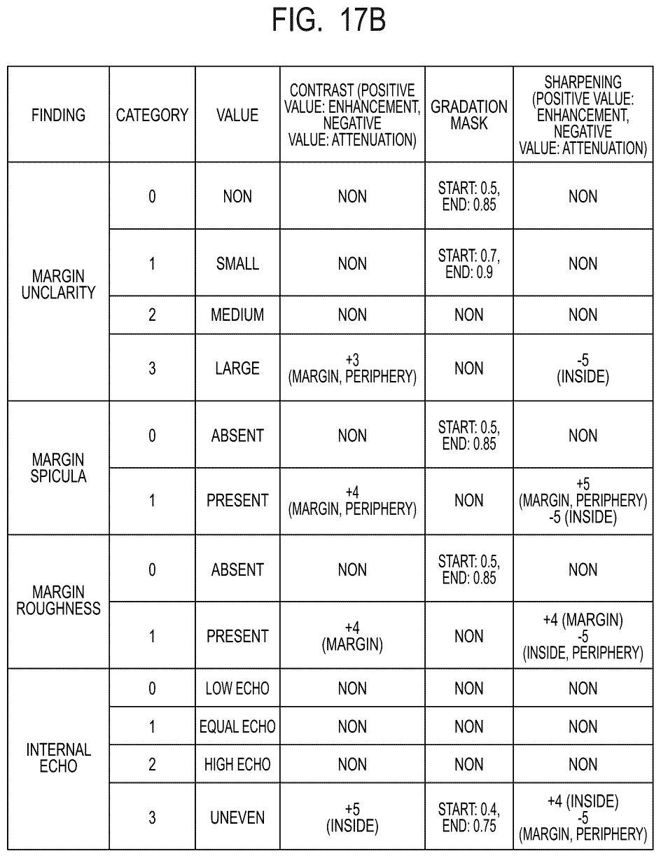

In step S16002, the input image acquisition unit 106 according to the fourth embodiment references the correspondence table illustrated in FIG. 17A as an example based on the category values of the degree of indistinctness of the tumor mass margin, the presence or absence of a spicula, the presence or absence of a roughness, and the internal echo pattern, each of which is the auxiliary information acquired in step S16001 (the auxiliary information acquisition unit 102), and acquires parameters for a modification process on the target image. Furthermore, the input image acquisition unit 106 modifies the target image with the acquired parameters and acquires the modified target image as the input image. In the present embodiment, the parameters are acquired based on four types of auxiliary information, respectively, modification is performed on the target image, and four input images are acquired.

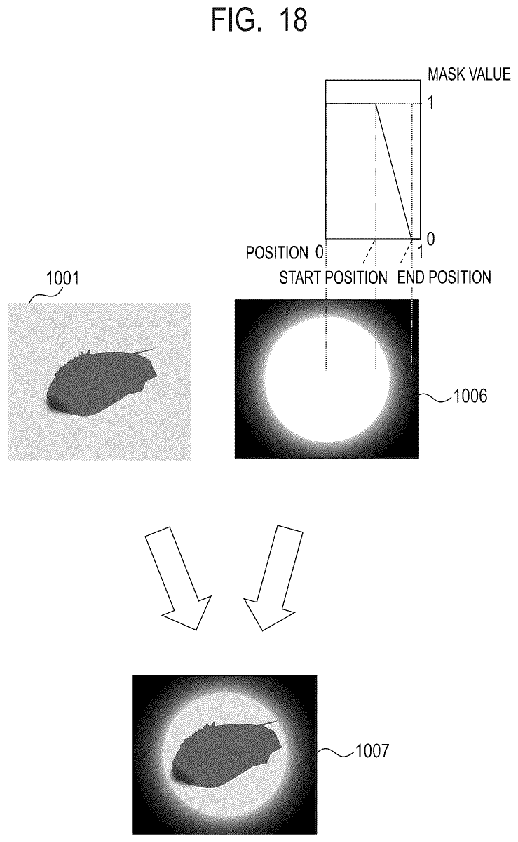

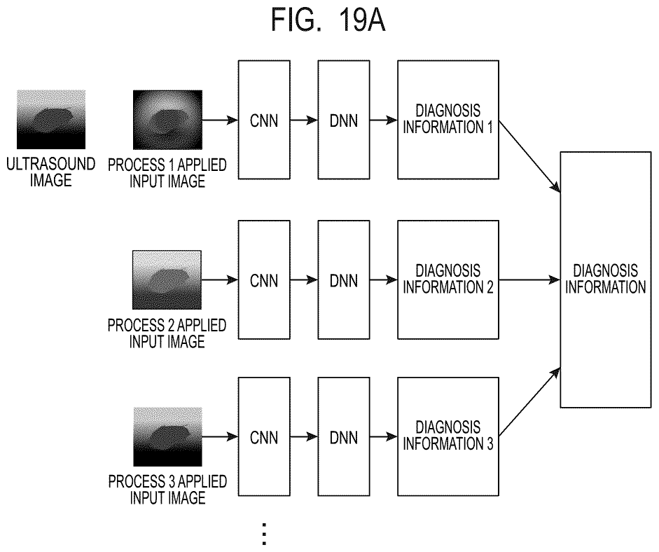

In the present embodiment, the process on the target image is to perform enhancement or attenuation of the contrast, application of a gradation mask, and enhancement or attenuation of the sharpness with a known filtering process. For the contrast and the sharpness, the intensity of enhancement or attenuation is acquired as the parameter, the intensity having a positive value corresponds to enhancement, and the intensity having a negative value corresponds to attenuation. Herein, when the sharpness is a negative value, a blurring process or the like can be applied to blur the image. The gradation mask 1006 is a mask that is darker in the periphery as illustrated in FIG. 18, and the start position and the end position of the gradation are aligned with the mask and acquired as the parameters. Herein, the start position and the end position of the gradation are expressed by relative values when the center is defined as 0 and the distance from the center to the image end in the lateral direction is defined as 1. Note that the method of creating the gradation mask 1006 is not limited to the above. For example, the value of the mask from the start position to the end position may be changed nonlinearly instead of being changed linearly.

Specifically, for example, when the category of roughness of the tumor mass margin, which is the auxiliary information, is 1 (present), a sharpening process is applied, and thereby an image with emphasized information on the tumor mass margin can be acquired as the input image. Further, for example, when the category of internal echo pattern is 3 (uneven), the gradation mask 1006 is applied to further enhance the contrast, and thereby a masked image 1007 with reduced information from the tumor mass margin to the periphery and with emphasized information on the inside of the tumor mass can be acquired as the input image.

Note that the above process is not necessarily required to be performed on all the four types of auxiliary information, and the target image may be modified with a parameter acquired based on any one type of auxiliary information to acquire one input image. Further, a plurality of parameters may be acquired based on one type of auxiliary information to acquire a plurality of input images. Further, one parameter may be acquired based on multiple types of auxiliary information to acquire one input image. Further, the auxiliary information is not limited to the degree of indistinctness of the tumor mass margin, the presence or absence of a spicula, the presence or absence of a roughness, and the internal echo pattern, and another image finding such as posterior features may be used as the auxiliary information.

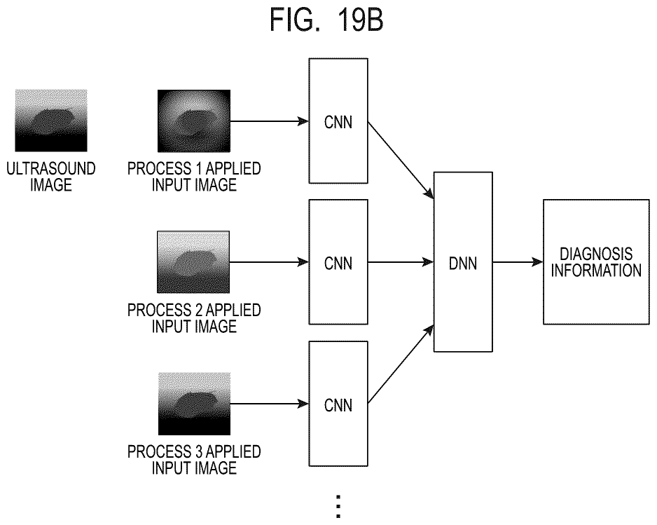

In step S16003, the diagnosis information inference unit 104 infers diagnosis information on the tumor mass included in the target image based on the input image acquired by the input image acquisition unit 106. In the present embodiment, the BI-RADS category of a tumor mass is inferred as the diagnosis information. To infer diagnosis information, a reasoner built in advance through deep learning or the like to infer diagnosis information based on images is used. In the present embodiment, as illustrated in FIG. 19A, a plurality of input images acquired in step S16002 are input to the reasoners, respectively, and the average value of the output diagnosis information (likelihoods of respective categories) is acquired as the final diagnosis information. Herein, reasoners built with different training data or schemes may be prepared for respective combinations of process parameters determined in accordance with types and categories of auxiliary information, and the reasoner to be used may be switched in accordance with the input image. Further, as illustrated in FIG. 19B as an example, the reasoners may be configured to output one piece of diagnosis information from a plurality of input images.

Note that the configuration of the diagnosis information inference unit 104 is not limited to the above, and the diagnosis information inference unit 104 may be configured to transmit an input image to an external server (not illustrated) that provides the same function as the diagnosis information inference unit 104, cause the server to infer diagnosis information, and acquire the diagnosis information.

The process described above makes it possible to apply a modification process in accordance with a finding in a medical image to a target image and use the image with the emphasized finding information for inference of diagnosis information. Accordingly, a feature of a finding in the medical image can be cased to be preferentially referenced when diagnosis information is inferred, and a reliable inference result can be obtained.

### Modified Example 1 for Fourth Embodiment

When the modification process is applied to the target image in step S16002, the region to which the process is applied may be restricted. For example, a correspondence table as illustrated in FIG. 17B is used to additionally acquire a region to be applied for respective processes. Herein, the region is any one of the inside, the margin, and the periphery of a tumor mass.

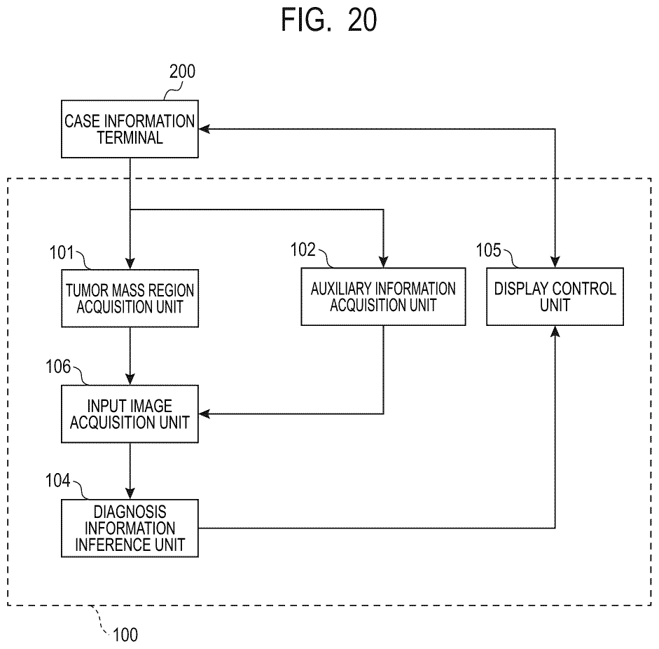

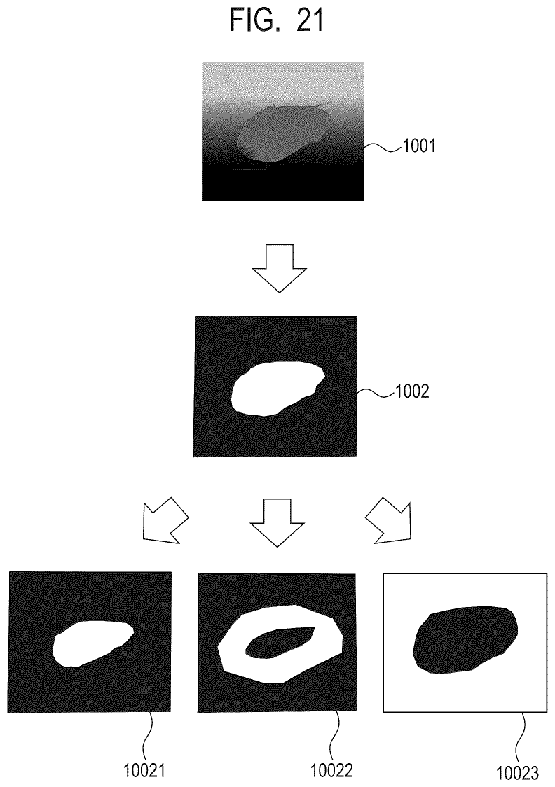

As illustrated in FIG. 20, the function configuration in Modified example 1 for the fourth embodiment is such that the tumor mass region acquisition unit 101 described in the first embodiment is added to the function configuration described in the fourth embodiment. A tumor mass internal region 10021, a tumor mass margin region 10022, and a tumor mass peripheral region 10023 are determined from the tumor mass region 1002 acquired by the tumor mass region acquisition unit 101 (illustrated in FIG. 21 as an example). In this case, the width of the region is adaptively determined in accordance with the size of the tumor mass, such as being determined in accordance with the width of the tumor mass in the medical image.

Accordingly, an image with more emphasized information on a finding can be input to a diagnosis reasoner. For example, when the category of the roughness of a tumor mass margin is 1 (present), by enhancing the sharpness of only the margin and attenuating the sharpness or the contrast of the inside and the periphery of the tumor mass, it is possible to generate an input image with more emphasized information on the margin.

### Modified Example 2 for Fourth Embodiment

The target region in Modified example 1 for the fourth embodiment is not limited to the inside, the margin, or the periphery of a tumor mass, and the finding region described above in the first embodiment may be used, for example.

The auxiliary information acquisition unit 102 in Modified example 2 for the fourth embodiment acquires a finding region as the auxiliary information and uses the acquired finding region as the target region. The finding region is, for example, a region of an indistinct portion of a tumor mass margin (5A02, illustrated in FIG. 5A as an example), a region of a spicula (5B02, illustrated in FIG. 5B as an example), a region of a rough portion (5C02, illustrated in FIG. 5C as an example), and a bright region inside a tumor mass (5D02, illustrated in FIG. 5D as an example), and another region such as a region of posterior features may be used.

A process is applied to a restricted region inside or outside a finding region, and thereby an image in which information on the finding is more emphasized can be input to a diagnosis reasoner.

### Modified Example 3 for Fourth Embodiment

The target region in Modified example 1 for the fourth embodiment is not limited to the inside, the margin, or the periphery of a tumor mass, and the reference region described above in the first embodiment may be used, for example.

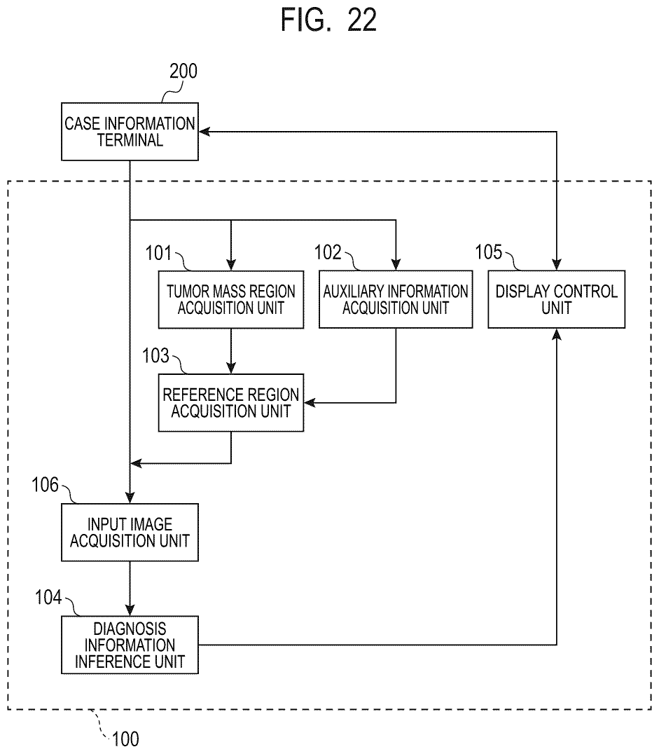

As illustrated in FIG. 22, the function configuration in Modified example 3 for the fourth embodiment is such that the tumor mass region acquisition unit 101 and the reference region acquisition unit 103 described in the first embodiment are added to the function configuration described in the fourth embodiment. The reference region acquisition unit 103 acquires a reference region (604, illustrated in FIG. 6 as an example) from a tumor mass region acquired by the tumor mass region acquisition unit 101 and a finding region acquired by the auxiliary information acquisition unit 102 and uses the acquired reference region as the target region.

A process is applied to a restricted region inside or outside a reference region, and thereby an image with more emphasized information intended to be referenced by a diagnosis reasoner can be input to the diagnosis reasoner.

### Fifth Embodiment

As the fifth embodiment, the present invention provides a medical image processing method including: a medical image acquisition step of acquiring a medical image including at least a region of a tumor mass; an auxiliary information acquisition step of acquiring auxiliary information including at least any one of A) image finding information, which is based on the medical image and is information about a predetermined image finding representing a nature of the tumor mass, and B) first diagnosis information, which is based on the medical image and is information about diagnosis of the tumor mass; and a diagnosis information inference step of, based on the medical image and on the auxiliary information, inferring second diagnosis information that is information about diagnosis of the tumor mass.

Further, a medical image processing program for causing a computer to perform the medical image processing method described above and a non-transitory storage medium storing the program in a computer readable form are provided as further embodiments.

### OTHER EMBODIMENTS

The embodiments described above are examples when provided to a system that, for the image capturing a mammary gland by using an ultrasound diagnosis device, infers diagnosis information on a tumor mass in an image. The same configuration can also be realized in other systems when these systems infer diagnosis information based on a medical image and can identify an image feature effective for the inference of diagnosis information.

For example, in a system that infers the grade of malignancy of a pulmonary nodule in a lung image captured by computed tomography (CT), the same effect can be obtained by acquiring a reference region so that an indistinct region or a rough region of the margin of the pulmonary nodule is included in the reference region and using the reference region for the inference.

### Modified Example

A medical image processing device in each embodiment described above may be realized as a single-unit device or may be realized in a form in which a plurality of devices are communicably combined to each other to perform the process described above, both of which are included in the embodiments of the present invention. The process described above may be performed by a common server device or server groups. A plurality of devices forming the medical image processing device and the image processing system can be communicated with each other at a predetermined communication rate and are not required to be present within the same facility or the same country.

The embodiments of the present invention include a form of providing a program of software that implements the functions of the embodiments described above to a system or a device and reading and executing codes of the provided program by a computer in the system or the device.

Therefore, the program code itself installed in a computer in order to realize the process according to the embodiments by the computer is also one of the embodiments of the present invention. Further, the OS or the like running on a computer performs a part of or the whole of the actual process based on instructions included in a program read by the computer, and the functions of the embodiments described above may be implemented by the process.

Forms in which the above embodiments are combined as appropriate are also included in the embodiments of the present invention.

According to the present invention, auxiliary information useful for inference of diagnosis information can be used for inference of diagnosis information. Accordingly, the diagnosis information can be inferred with high reliability.

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2023-173727, filed Oct. 5, 2023, and Japanese Patent Application No. 2024-112578, filed Jul. 12, 2024, which are hereby incorporated by reference herein in their entirety.

What is claimed is:

1. A medical image processing device comprising:
a medical image acquisition unit that acquires a medical image including at least a region of a tumor mass;
an auxiliary information acquisition unit that acquires auxiliary information including at least any one of
A) image finding information, which is based on the medical image and is information about a predetermined image finding representing a nature of the tumor mass, and
B) first diagnosis information, which is based on the medical image and is information about diagnosis of the tumor mass; and
a diagnosis information inference unit that infers second diagnosis information, which is information about diagnosis of the tumor mass, in response to input of an image generated based on the medical image and on the auxiliary information.

2. The medical image processing device according to claim 1, wherein the diagnosis information inference unit has a reference region acquisition unit that, based on the medical image and on the auxiliary information, acquires a reference region image in the medical image as the generated image, and the diagnosis information inference unit infers the second diagnosis information based on the reference region image.

3. The medical image processing device according to claim 2, wherein the reference region acquisition unit acquires a region enlarged from or a region reduced from the region of the tumor mass as a reference region based on the auxiliary information.

4. The medical image processing device according to claim 3, wherein the reference region acquisition unit determines a ratio of enlargement or reduction of the region of the tumor mass based on the auxiliary information.

5. The medical image processing device according to claim 2, wherein the reference region acquisition unit further adjusts the sharpness of a boundary of an acquired reference region based on the auxiliary information.

6. The medical image processing device according to claim 2,
wherein the reference region acquisition unit includes
a first reference region acquisition unit that acquires a first reference region in the medical image based on the medical image and on the auxiliary information, and
a second reference region acquisition unit that acquires a second reference region, which differs from the first reference region, based on the medical image and on the auxiliary information, and
wherein the diagnosis information inference unit infers the second diagnosis information based on the medical image, on the first reference region, and on the second reference region.

7. The medical image processing device according to claim 6, wherein the diagnosis information inference unit has
a first region-related diagnosis information inference unit that infers first region-related diagnosis information, which is diagnosis information about the first reference information, based on the medical image and on the first reference region, and
a second region-related diagnosis information inference unit that infers second region-related diagnosis information, which is diagnosis information about the second reference region, based on the medical image and on the second reference region, and
infers the second diagnosis information based on the first region-related diagnosis information and on the second region-related diagnosis information.

8. The medical image processing device according to claim 1 further comprising an input image acquisition unit that acquires, as an input image that is the generated image, an image obtained by modifying a pixel value of the medical image based on the auxiliary information,
wherein the diagnosis information inference unit infers the second diagnosis information based on the input image.

9. The medical image processing device according to claim 1,
wherein the first diagnosis information includes information about at least any one of
a result of pathological diagnosis on the tumor mass, and
a grade of malignancy of the tumor mass based on a result of image diagnosis on the tumor mass, and
wherein the second diagnosis information includes information about the grade of malignancy of the tumor mass.

10. The medical image processing device according to claim 1,
wherein the auxiliary information includes at least A) the image finding information, and
wherein the image finding information includes information about an image finding region that is a region in the medical image, the image finding being present in the region.

11. The medical image processing device according to claim 1,
wherein the auxiliary information includes at least A) the image finding information, and
wherein the image finding information includes information about the presence or absence, a type, or a degree of the image finding.

12. The medical image processing device according to claim 1,
wherein the auxiliary information includes at least A) the image finding information, and
wherein the image finding information includes at least any one of information about distinctness of a margin of the tumor mass, information about roughness of a margin of the tumor mass, and information about the presence or absence of a linear opacity of a margin of the tumor mass.

13. The medical image processing device according to claim 1 further comprising a tumor mass region acquisition unit that acquires a region of a tumor mass included in a target image based on the medical image.

14. The medical image processing device according to claim 2 further comprising a tumor mass region acquisition unit that acquires a region of a tumor mass included in a target image based on the medical image,
wherein the reference region acquisition unit acquires a reference region based on a tumor mass region acquired by the tumor mass region acquisition unit and on the auxiliary information.

15. A medical image processing method comprising:
a medical image acquisition step of acquiring a medical image including at least a region of a tumor mass;
an auxiliary information acquisition step of acquiring auxiliary information including at least any one of
A) image finding information, which is based on the medical image and is information about a predetermined image finding representing a nature of the tumor mass, and
B) first diagnosis information, which is based on the medical image and is information about diagnosis of the tumor mass; and
a diagnosis information inference step of inferring second diagnosis information, which is information about diagnosis of the tumor mass, in response to input of an image generated based on the medical image and on the auxiliary information.

16. A non-transitory storage medium storing a medical image processing program in a computer readable form, the medical image processing program being for causing a computer to perform the medical image processing method according to claim 15.

17. The medical image processing device according to claim 8, wherein the input image acquisition unit acquires a medical image processed so as to emphasize at least any one of an inside, a margin, or a periphery of the tumor mass in accordance with a result of the auxiliary information.

18. The medical image processing device according to claim 8, wherein the input image acquisition unit acquires a medical image processed so as to reduce at least any one of an inside, a margin, or a periphery of the tumor mass in accordance with a result of the auxiliary information.

19. The medical image processing device according to claim 8,
wherein the auxiliary information includes at least A) the image finding information,
wherein the image finding information includes information about an image finding region that is a region in the medical image, the image finding being present in the region, and
wherein the input image acquisition unit acquires a medical image processed so as to emphasize the inside or the outside of the image finding region.

20. The medical image processing device according to claim 8,
wherein the auxiliary information includes at least A) the image finding information,
wherein the image finding information includes information about an image finding region that is a region in the medical image, the image finding being present in the region, and
wherein the input image acquisition unit acquires a medical image processed so as to reduce the inside or the outside of the image finding region.
